(19) United States
(12) Patent Application Publication
(10) Pub. No.: US 2007/0026390 A1
(43) Pub. Date: Feb. 1, 2007

(54) Development of diagnostic kit against the recombinant coat protein of prunus necrotic ringspot virus

(75) Inventors: Saurabh Kulshrestha, Palampur (IN); Vipin Hallan, Palampur (IN); Gaurav Raikhy, Palampur (IN); Aijaz Asghar Zaidi, Palampur (IN)

Correspondence Address: MERCHANT & GOULD PC, P.O. BOX 2903, MINNEAPOLIS, MN, 55402-0903, US

(73) Assignee: Council of Scientific and Industrial Research, Rafi Marg (IN)

(21) Appl. No.: 11/367,625

(22) Filed: Mar. 3, 2006

(30) Foreign Application Priority Data
Jul. 25, 2005 (IN) 1979/DEL/2005

(51) Int. Cl. C12Q 1/70; C12Q 1/68; C07H 21/04

(52) U.S. Cl. 435/5; 435/6; 536/23.72

(57) ABSTRACT

The present invention deals with a set of primers of sequence ID 1: upstream primer AACTGCAGATGGTTTGCCGAATTTGCAA and sequence ID 2: downstream primer GCTCTAGACTAGATCTCAAGCAGGTC useful for detection of Prunus necrotic ringspot virus in plants. It also relates to a method for detection of Prunus necrotic ringspot virus in plants by using said primers. Further the invention also relates to a diagnostic kit useful for detection of coat protein of Prunus necrotic ringspot in plants.

### FIELD OF INVENTION

The present invention relates to a primers useful for detection of Prunus necrotic ringspot virus in plants.

More particularly this invention relates to a method for detection of Prunus necrotic ringspot virus in plants by using a primers useful for detection of Prunus necrotic ringspot virus in plants. The present invention also relates to a diagnostic kit useful for detection of coat protein of Prunus necrotic ringspot in plants.

### BACKGROUND OF INVENTION

Prunus necrotic ringspot virus (PNRSV), an ilarvirus, has a wide host range especially on important commercial crops like rose (Garden roses, cut flower roses and essential oil bearing roses) and on important crops like apple, peach, hop, apricot, almond and plum. PNRSV was detected in roses and other commercial crops at the Institute of Himalayan Bioresource Technology (IHBT), Palampur by enzyme linked immunosorbent assay (ELISA) and reverse transcription-polymerase chain reaction (RT-PCR). PNRSV coat protein and movement protein genes have been cloned and sequenced at IHBT, Palampur. Primer pairs were designed for the amplification of complete coat protein and movement protein genes of PNRSV. The sequences of the designed primers were submitted to EMBL database (PNRSVCP dn-AJ619983, PNRSVCP up-AJ619984, PNRSVMP up-AJ697738 and PNRSVMP dn-AJ697739). Using these designed primers, the complete coat protein and movement protein genes of rose isolate of PNRSV were successfully amplified, cloned and sequenced. The sequences of complete coat protein and movement protein genes were submitted to EMBL database. (CP-AJ619958, MP-AJ697737). PNRSV has been reported to be more prevalent in rose than other viruses (Wong, S. M., Horst, R. K., Langhans, R. W. (1988), Symptomology and occurrence of Apple mosaic and Prunus necrotic ringspot viruses on rose in New York. Acta Horticulturae 234: 437-450) and also in Prunus (Moury, B., Cardin, L., Onesto, J. P., Candresse, T., Poupet, A. (2001), Survey of Prunus necrotic ringspot virus in rose and its variability in rose and Peach spp. Phytopathology 91:84-91). PNRSV is ubiquitous in rose and adversely affecting the quality and quantity of flowers (the flower quantity was decreased up to 25-30% as compared to the healthy plants), stem length and vase life (Moran, J. R., Faragher, J. D., Baker, D. M. (1988), The effect of Prunus necrotic ringspot virus on production and quality of rose flowers. Acta Horticulturae. 234:429-434), (Thomas, B. J. (1981), The effects of Prunus necrotic ringspot virus on field grown roses. Annals of Applied Biology 100: 129-134). It was found that only 20% of the graft unions with PNRSV infected scions were successful, as compared to 90% success with healthy scions (Stein, A., Levy, S., Loebenstein, G. (1988), Effects of Prunus necrotic ringspot virus on graft union and Flower quality of glasshouse grown roses in Israel. Acta Horticulturae 234:435). On peach PNRSV isolates did not display visible symptoms on the leaves but twig and branch withering was induced as well as death of whole trees. It decreases the leaf and flower buds as well as the nodes of the infected trees by 66-96%. Leaf size was decreased by 14-80%. The trunk and limb circumference of the trees was decreased, too (42-71%) (Topchiiska, M. L. (1983), Effect of Prunus necrotic ringspot virus (PNRSV) and Prune dwarf virus (PDV) on some biological properties of peach*. Acta Horticulturae 130:307-312). In case of sour cherry PNRSV-infected trees yielded 744.9 gm fruit whereas the healthy ones, 828.5 gm fruit per tree. Growth was not significantly affected by PNRSV (Ramsdell, D. C., Bird, G. W., Adler, V. A., Gillett, J. M. (1992), Effects of Tomato ringspot virus and Prunus necrotic ringspot virus alone and in combination on the growth and yield of ‘Montmorency’ sour cherry. Acta Horticulturae 309:111-114). PNRSV was also detected in begonia (Verma, N., Hallan, V., Ram, R., Zaidi, A. A., (2002), Detection of Prunus necrotic ringspot virus in begonia by RT-PCR. Plant Pathology, 51: 800) and in geranium (unpublished report).

Rose is one of the important commercially grown cut flower crop round the world. It ranks among the top three flowers of the world. Since it is being propagated through cuttings, viral infection tends to be carried from generation to generation through cuttings. For maintaining quality of germplasm and minimizing the infection of viruses to different cultivars, proper diagnostics and control for viral diseases are not only highly desirable but essential for improving crop productivity.

Thomas (Thomas, B. J. (1981), Studies on rose mosaic disease in field grown roses produced in The United Kingdom. Annals of Applied Biolology 98:419-429) observed that chlorotic line patterns, ringspots, or mottles are generally induced by the cherry serotype of Prunus necrotic ringspot virus (PNRSV), while an apple serotype (Apple mosaic virus, ApMV) causes stunted, distorted and puckered leaflets on roses (Thomas, B. J. (1984), Rose mosaic disease: symptoms induced in roses by graft inoculation with both Peach necrotic ringspot and Apple mosaic viruses. Plant Pathology 33:155-160).

Traditional diagnosis of plant viruses requires bioassaying through an indicator plant, symptom observation, host range determination, and virus particle morphology and vector relations. However, progress in molecular biology, biochemistry and immunology has lead to the development of many new accurate, rapid and less labour-intensive methods of virus detection. There are various diagnostic techniques available in the field of virology like precipitation tests, agglutination tests, fluorescent antibody test, enzyme-linked immunosorbent assay, electron microscopy, immune electron microscopy, dot immunobinding assay, tissue blotting assay, western blotting, and nucleic acid hybridization with radiolabeled and non radiolabeled probes and polymerase chain reaction based detection.

The immunodiffusion in gels, the latex flocculation assay were used for the detection of PNRSV. The latex test was upto 250 times more sensitive than gel immunodiffusion (Thomas, B. J. (1980), The detection by serological methods of viruses infecting the rose. Annals of Applied Biology 94: 91-101).

Enzyme linked immunosorbent assay (ELISA) and other modified forms of ELISA have been extensively used for the detection of PNRSV from rose. It is able to detect and serotype PNRSV in various tissues and at various times through the growing season when assessed for several rosaceous hosts of this virus. In all the hosts studied (apple, cherry, plum and rose) flowers and flower parts, excepting rose sepals, were found to be good sources of antigen. Strains of the ‘C’ (cherry) serotype were generally more readily detectable in leaf samples in spring than later in the season and in autumn more readily detectable than in midsummer, though some isolates were most easily detected in autumn. Strains of the ‘A’ (apple mosaic) serotype were best detected in spring and many were undetectable later in the season. (Barbara, D. J. (1981), Detecting Prunus necrotic ringspot virus in rosaceous hosts by enzyme-linked immunosorbent assay. Acta Horticulturae 94:329-332). Wong and Horst (Wong, S. M., Horst, R. K. (1988), Comparison of antigen and antibody-coated enzyme linked immunosorbent assay procedures for the detection of three isolates of purified Apple mosaic virus or Prunus necrotic ringspot virus. Acta Horticulturae 234:249-256) established an ELISA system to detect PNRSV in rose and compared the sensitivity of antigen and antibody coated ELISA to detect PNRSV in crude extracts. The antibody coated ELISA (DAS-ELISA) procedure was found to be the most suited for the detection of PNRSV in crude extracts and antigen coated ELISA (Direct ELISA) was not successful in the detection of PNRSV in crude plant extracts. ELISA is the most preferred method for the mass screening of PNRSV in infected stone fruits like almond, apricot, cherry, nectarine, peach, plum etc. (Salem, N., Mansour, A., Al-Musa, A., Al-Nsour, A. (2003), Incidence of Prunus necrotic ringspot virus in jordan. Phytopathologica Mediterranea 42:275-279) (Scott, S. W., Barnett, O. W., Burrows, P. M. (1989), Distribution and prevalence of Prunus necrotic ringspot virus in peach orchards of South Carolina. Acta Horticulturae 235:137-142) (Scott, S. W., Walker Miller, R., Bachman, E. J. (1992), Evidence for the spread of Prunus necrotic ringspot virus and the presence of Prune dwarf virus in selected peach orchards in South Carolina. Acta Horticulturae 309:73-78). The concentration of PNRSV varies from time to time, plant to plant and even plant part to plant part, Therefore, type of sample and sampling time are critical for the reliable detection of PNRSV in the infected plants as they appear to be healthy (Dal Zotto, A., Nome, S. F., Di Rienzo, J. A., Docampo, D. M. (1999), Fluctuations of Prunus necrotic ringspot virus (PNRSV) at various stages of peach cultivars. Plant Disease 83:1055-1057). Petals and leaf tissues collected early in the season will yield the most reliable results (Bertozzi, T., Alberts, E., Sedgley, M. (2002), Detection of Prunus necrotic ringspot virus in almond: effect of sampling time on the efficiency of serological and biological indexing methodologies. Australasian Journal of Plant Pathology 42:207-210). PNRSV were generally more readily detectable in leaf samples in spring than later in the season and in autumn more readily that in mid winter (Barbara, D. J. (1980), Detecting Prunus necrotic ringspot virus in rosaceous hosts by enzyme linked immunosorbent assay. Acta Phytopathologica-Academiae-Scientiarum-Hungaricae 15: 329-332). These seasonal fluctuations in the virus concentration did not follow the same pattern in all cultivars. It is, therefore, impossible to distinguish between infected and healthy trees on the basis of one single sampling time for all cultivars (Salem, N., Mansour, A., Al-Musa, A., Al-Nsour, A. (2003), Seasonal variation of Prunus necrotic ringspot virus concentration in almond, peach, and plum cultivars. Phytopathologica Mediterranea 42:155-160).

PNRSV has a very wide host range, has many isolates with different biological, serological and molecular properties. Polyclonal antibodies failed to differentiate among these isolates of PNRSV. In order to differentiate the various isolates of PNRSV, monoclonal antibodies were used and the virus showed high serological variability. Thirty four serogroups were identified with monoclonal antibodies 64% of which were host specific and 67% being country specific (Mytra, A., Terlizzi, B. D., Boscia, D., Choueiri, E., Gatt, M., Gavriel, I., Caglayan, K., Varveri, C., Zeramdini, H., Aparico, F., Pallas, V., Savino, V. (2001), Serological characterization of Mediterranean Prunus necrotic ringspot virus isolates. Journal of Plant Pathology 83(1):45-49). PNRSV is a very labile virus, its longevity in vitro (LIV) is only 6-18 hours (Brunt, A. A., Crabtree K., Dallwitz, M. J., Gibbs, A. J., Watson, L. (1996): Viruses of plants, CAB International, pp. 1047-1049 University Press, Cambridge) and is difficult to purify in good amount for the purpose of antibody production. By the advancement of technology, systems are now available for the expression of coat protein gene of virus in Escherichia coli (E.coli). Petrizk and his coworkers (Petrzik, K., Mraz, I., Kubelkova, D. (2001), Preparation of recombinant coat protein of Prunus necrotic ringspot virus. Acta Virologica 45:61-63) expressed the coat protein gene of PNRSV in E.coli.

Similar to ELISA, Electron microscopy in various forms is also used for the detection of viruses in plants (Corbett, M. K. (1974), Detection of viruses and diagnosis of plant viral diseases by electron microscopy. Acta Horticulturae 36:141-188). Serologically specific electron microscopy (SSEM) or Immune electron microscopy (IEM) was also used to detect PNRSV. SSEM and ELISA were, respectively, up to 1000 and 200 times more sensitive that latex test. Gel immunodiffusion and latex tests failed to detect PNRSV in infected rose, although ELISA could reliably detect it (Thomas, B. J. (1980), The detection by serological methods of viruses infecting the rose. Annals of Applied Biology 94: 91-101). Electron microscopy (DIP, ISEM and decoration) was used for the detection of PNRSV in almond, plum and peach trees also (Boullia, M., Marrakchi, M. (2001), Detection and characterization of stone fruit virus disease in tunisia. Phytopathologica Mediterranea 40: 125-136).

Immunogold-silver labelling technique was used for the localization of PNRSV in peach tissue. Labelling was concentrated in the cortical parenchyma tissue. Occasionally, a few cells in the xylem parenchyma were also specifically labelled. Labelling was never detected in epidermis, vascular tissue and cambium or in pith. Almost all-cortical parenchyma cells of the petiole and stem, but only a few root cells showed silver labelling. Within the parenchyma cells the labelling was spread throughout the cytoplasm (Giunchedi, L., Poggi Pollini, C. (1989), Localization of Prunus necrotic ringspot virus antigen in peach tissue by immunogold-silver labelling. Acta Horticulturae 235:191-196).

During the last decade, PCR has been widely used with varying degree of modification for the detection of viral genome in infected plants. When reverse transcription polymerase chain reaction (RT-PCR) and ELISA were compared for the detection efficiency, RT-PCR was found to be more sensitive than ELISA in case of PNRSV (Mekuria, G., Ramesh, S. A., Alberts, E., Bertozzi, T., Wirthensohn, M., Collins, G., Sedgley, M. (2003), Comparision of ELISA and RT-PCR for the detection of Prunus necrotic ringspot virus and Prune dwarf virus in almond (Peach dulcis). Journal of Virological Methods 114:65-69). The RT-PCR was used for the detection of PNRSV by the amplification of two different pairs of primers yielding a short and long product, respectively. The relative amount of short product was higher than the longer product. PNRSV was detected better in plant tissues with a low virus concentration (e.g. dormant plants) by the amplification of short PCR product, whereas the long product at higher virus titers (Rosner, A., Maslenin, L., Spiegel, S. (1997), The use of short and long PCR products for improved detection of Prunus necrotic ringspot virus in woody plants. Journal of Virological Methods 67:135-141). Multiplex RT-PCR was also used for the detection of different viruses in a single reaction. This technique was very sensitive for the detection of more that one virus in mixed infected plant (Mekuria, G., Ramesh, S. A., Alberts, E., Bertozzi, T., Wirthensohn, M., Collins, G., Sedgley, M. (2003), Comparison of ELISA and RT-PCR for the detection of Prunus necrotic ringspot virus and Prune dwarf virus in almond (Peach dulcis). Journal of Virological Methods 114:65-69). Immuno capture RT-PCR (IC-RT-PCR) alone and in combination with nested PCR is even more sensitive for the PNRSV. Inhibitory effects and inconsistencies of the standard IC-RT-PCR were overcome by IC-RT-PCR nested PCR. The later improved the detection by three orders of magnitude compared with DAS-ELISA for the detection of PNRSV in leaves (Helgurea, P. R., Toborda, R., Docampo, D. M., Ducasse, D. A. (2001), Immunocapture reverse transcription-polymerase chain reaction combined with nested PCR greatly increases the detection of Prunus necrotic ringspot virus in peach. Journal Virological Methods 95:93-100). PNRSV exits as a number of biologically distinct variants, which differs in host specificity, serology and pathology. The use of type-specific primers that amplify a portion of coat protein gene in a multiplex PCR protocol permits rapid detection and discrimination among different PNRSV strains (Hammond, R. W., Crosslin, J. M., Pasini, R., Howell, W. E., Mink, G. I. (1999), Differentiation of closely related but biologically distinct cherry isolates of Prunus necrotic ringspot virus by polymerase chain reaction. Journal of Virological Methods 80:203-212).

Non-isotopic molecular hybridization technique was also used for the detection of PNRSV (Saade, M., Aparicio, F., Sanchez-Navarro, J. A., Herranz, M. C., Myrta, A., Di-Terlizzi, B., Pallas, V. (2000), Simultaneous detection of the three ilarviruses affecting stone fruit trees by nonisotopic molecular hybridization and multiplex reverse-transcription polymerase chain reaction. Phytopathology 1330-1336). In an another technique, partial nucleic acid sequences of different viruses (including PNRSV) were cloned in tandem allowing the synthesis of a unique riboprobe called ‘Polyprobe” for the detection of a number of viruses simultaneously (Herranz, M. C., Sanchez-Navarro, J. A., Aparicio, F., Pallas, V. (2004), Simultaneous detection of six stone fruit viruses by non-isotopic molecular hybridization using a unique riboprobe or ‘Polyprobe’. Journal of Virological Methods 124:49-55).

Thus DAS-ELISA, EM, IEM, RT-PCR, IC-RT-PCR and hybridization are the suitable techniques to detect PNRSV in rose and other crops. RT-PCR, IC-RT-PCR and nucleic acid hybridization are the sensitive tools to detect the virus but these require sophisticated instruments which are costly too. So, uptill now, ELISA have been extensively used for the diagnosis of viruses infecting rose and other commercially important crops like stone fruits, hops, geranium etc. because these are quick methods, easy to perform, can be used even in field conditions and are cost effective as well. So, these can be exploited in the form of a diagnostic kit.

### OBJECTS OF THE INVENTION

The main object of the present is to provide primers useful for detection of Prunus necrotic ringspot virus in plants.

Another object of the present invention is to provide a method for detection of Prunus necrotic ringspot virus in plants by using designed primers useful for detection of Prunus necrotic ringspot virus in plants.

Still another object of the present invention is to provide a diagnostic kit useful for detection of coat protein of Prunus necrotic ringspot virus in plants.

### SUMMARY OF THE INVENTION

The present invention relates to a method for detection of Prunus necrotic ringspot virus in plants using desined primers of
Sequence ID 1: upstream primer AACTGCAGATGGTTTGCCGAATTTGCAA
Sequence ID 2: downstream primer GCTCTAGACTAGATCTCAAGCAGGTC;

It also relates to a diagnostic kit useful for detection of coat protein of Prunus necrotic ringspot in plants comprising:
a) polyclonal antibodies against Prunus necrotic ringspot virus coat protein in plants
b) conjugate labeled with alkaline phosphatase;
c) coating buffer;
d) extraction buffer;
e) ECI buffer;
f) PNP buffer.

### DETAILED DESCRIPTION OF THE INVENTION

For the purpose of development of a diagnostic kit, the complete coat protein of PNRSV was amplified from rose using designed primers that would amplify the complete coat protein gene of PNRSV. The complete coat protein gene was then cloned and sequenced to confirm the identity of the amplicon. It was then cloned inframe in pGEX-2TK and E.coli strain BL21 was transformed with the recombinant pGEX-2TK vector containing the coat protein gene of PNRSV. The expression conditions like induction temperature and IPTG concentration were standardized for optimal expression of PNRSV coat protein. The optimal expression was found at 30° C. and at ImM IPTG concentration, the expressed protein was then purified from 250 ml culture. Two Rabbits were then immunized thrice at weekly intervals with 100 μg of purified protein with Freund's complete adjuvant (Meenu Katoch, A. A. Zaidi and Raja Ram. 2002. Development of diagnostic kit for the detection of Bean yellow mosaic virus. Patent file no 76/NF/2002, Pending) intramuscularly and subcutaneously and once intravenously with same amount of protein in Freund's incomplete adjuvant. Rabbit was bled after 15 days, antibodies were purified from the serum and stored at −20 ° C.

Accordingly the present invention provides primers useful for detection of Prunus necrotic ringspot virus (PNRSV) in plants, comprising the following sequence:
Sequence ID 1: upstream primer AACTGCAGATGGTTTGCCGAATTTGCAA
Sequence ID 2: downstream primer GCTCTAGACTAGATCTCAAGCAGGTC

Further, the present invention also provides a method for detection of Prunus necrotic ringspot virus (PNRSV) in plants, wherein the said method comprising the steps of:
a) providing a purified coat protein of PNRSV by using designed primers of
   Sequence ID 1: upstream primer AACTGCAGATGGTTTGCCGAATTTGCAA
   Sequence ID 2: downstream primer GCTCTAGACTAGATCTCAAGCAGGTC;
b) preparing polyclonal antibodies against PNRSV coat protein obtained from step (a); c) performing direct antibody sandwich enzyme linked immunosorbent assay (DAS ELISA) for detection of PNRSV.

In an embodiment of the present invention, the complete coat protein of PNRSV is amplified using designed primers having a
sequence ID 1: upstream primerAACTGCAGATGGTTTGCCGAATTTGCAA
sequence ID 2: downstream primerGCTCTAGACTAGATCTCAAGCAGGTC

In another embodiment of the present invention, the complete coat protein of PNRSV with sequence ID 3:
MVCRICNHTHAGGCRSCKKCHPNDALVPLRAQQRVANNPNRNRNPNRVSSGMGPAVR PQPVVKTTWTVRGPNVPPRIPKGYVAHNHREVMTTEAVKYLSIDFTTTLPQLMGQNLTL LTVIVRMNSMSSNGWIGMVEDYKVDQPDGPNALSRKGFLKDQPRGWQFEPPSDLDFDT FARTHRVVIEFKTEVPAGAKVLVRDLYVVVSDLPRVQIPTDVLLVDEDLLEI is cloned in pGEX-2TK followed by transformation using E-coli strain BL 21.

Further in an embodiment of the present invention, the optimal expression of PNRSV coat protein is checked with 0.5-0.9 mM IPTG concentration at about temperature 30 degree C. for 3-3.5 h

Still in an embodiment of the present invention, obtained coat protein of PNRSV is sequenced by known sequencing methods.

Still in an embodiment of the present invention, the purification of PNRSV coat protein is carried out by the known method.

Still an embodiment of the invention, the immunization in rabbits are carried out three times with purified coat protein of PNRSV obtained from step 1(a) and Freund's complete adjuvant in the ratio of 1:1 at weekly intervals.

Still in an another embodiment of the present invention, the route for immunization may be intramuscularly, subcutaneously or intravenously.

Yet in an another embodiment of the present invention, the rabbits are bled after 14 to 15 days to obtain polyclonal antibodies against PNRSV coat protein.

Yet in an another embodiment of the present invention, the polyclonal antibodies against PNRSV coat protein are purified from the serum by known methods.

Yet another embodiment of the present invention, the microtiter plates are coated with polyclonal antibodies diluting in a coating buffer in a ratio ranges from 1:500-1:1000 followed by 4-5 times washing with PBS-T.

Yet another embodiment of the present invention, the test samples are prepared in microtiter plates by macerating infected leaf tissue from plant with extraction buffer followed by dilution from 1×- 1/160× of the original antigen.

Yet in another embodiment of the present invention, the microtiter plate is incubated overnight at about 37° C. followed by washing to allow coating of antigen in the wells.

Yet in an another embodiment of the present invention, the antibody conjugate in ECI buffer is added in the ratio ranges between 1:500 to 1:1000 for a period of about 4 hrs at about 37° C. followed by washing with PBS-T

Yet in an another embodiment of the present invention, about 10 μl of about 1 mg/ml p-nitrophenyl phosphate solution in PNP buffer is added in the mix.

Yet in an another embodiment of the present invention, the reaction is terminated by adding about 50 μl of about 3M NaOH after 15-20 min to obtain yellow color product.

Yet in an another embodiment of the present invention, the color product is antigen and antibody conjugate.

Yet in an embodiment of the present invention, the absorbance of colored product is measured at 405 nm for detection of Prunus necrotic ringspot virus.

Further, the present invention also provides a diagnostic kit useful for detection of coat protein of Prunus necrotic ringspot virus comprising:
a) polyclonal antibodies against Prunus necrotic ringspot virus coat protein in plants;
b) conjugate labeled with alkaline phosphatase;
c) coating buffer;
d) extraction buffer;
e) ECI buffer;
f) PNP buffer.

A part of antibodies and conjugate were kept in refrigerator (4-10° C.), whereas the other part was kept at room temperature (20-35° C.) to be used for studies later on. Results were found positive every time in first part, whereas using other part, results were positive only for 2-3 months and whole year at room temperature and at 4° C. in refrigerator respectively

The following examples are given by way of illustration of the present invention and should not be construed to limit the scope of present invention.

### EXAMPLE 1

Detection of PNRSV from Rose:

Different cultivars of rose were checked using DAS-ELISA as described below. At the same time they were also checked by a reference kit DSMZ (Deutsche Sammlung von Mikroorganismen und Zellkulturen GmbH, Germany).

Direct Antibody Sandwich ELISA:

1. Plates (Nunc Immuno TM Plant, Denmark) were coated with 100 μl of polyclonal antibodies (diluted 1:500-1000) in coating buffer and plates were incubated at 37° C. for 4 hrs in a humid box.
2. The plates were washed five times with PBS-T for 2 min each.
3. Test samples were prepared by macerating infected leaf tissue 1 gm/2 ml in extraction buffer. Several dilutions were made corresponding to 1×- 1/160× dilution of the original antigen and 100 μl of the diluted antigen was pipetted into the wells of microtitre plate as per the loading diagram and incubated overnight at 4° C. in a humid box to allow coating of antigen in the wells.
4. The plate was washed five times with PBS-T for 2 min each.
5. Antibody conjugate (diluted 1:500-1000) in ECI buffer was added into the wells (100 μl/well). Plates were incubated for 4 hrs at 37 ° C. in a humid box.
6. The plate was washed again for five times with PBS-T for 2 min each.
7. After washing, the wells were filled with 100 μl solution of 1 mg/ml p-nitrophenyl phosphate made in PNP buffer.
8. After appropriate colour development (30 min-2 hrs), the reaction was terminated by adding 50 μl of 3M NAOH to each well.
9. Positive and negative controls were also made on the same plate. Absorbance at 405 nm was measured for complete ELISA plate with a flow ELISA microplate reader. The reaction was considered positive if absorbance was observed to be greater that 0.1, which was at least three times the background of healthy control.

Results were positive in the positive control sample, whereas negative in the negative control sample.
Coating buffer (0.05M per liter): 1.59 g sodium carbonate and 2.93 g sodium bicarbonate, pH 9.6
PBS buffer: 20 mM sodium phosphate pH 7.4 and 150 mM NaCl
PBS-T buffer: 20 mM sodium phosphate pH 7.4; 150 mM NaCl and 0.05% (v/v) Tween 20
Extraction buffer: 1.3 g sodium phosphate (anhydrous), 20 g Polyvinylpyrrolidone (PVP) MW 24-40,000, 0.2 g sodium azide, 2.0 g powdered egg albumin grade II and 20.0 g Tween-20 were dissolved in 1000 ml 1×PBST and pH was adjusted to 7.4
ECI Buffer: 2.0 g BSA, 20.0 g PVP 24-40,000 and 0.2 g sodium azide were dissolved in 1000 ml 1×PBST and pH was adjusted to 7.4

PNP buffer: 0.1 g magnesium choride, 0.2 g sodium azide and 97 ml diethanolamine were dissolved in 800 ml distilled water, pH was adjusted to 9.8 and volume was made to 1000 ml.

TABLE 1
ELISA results of different cultivars of rose
using reference kit and test kit

| | | ELISA using | |
|---|---|---|---|
| S.No. | Cultivar of Rose | Reference kit | ELISA using test kit |
| 1. | Careless love | + | + |
| 2. | Blue moon | + | + |
| 3. | Maiden drought | + | + |
| 4. | Golden giant | + | + |
| 5. | Oklahoma | + | ++ |
| 6. | Red master piece | + | + |
| 7. | Kiss of love | + | + |
| 8. | Happiness | + | + |
| 9. | Sabaien | + | + |
| 10. | Queen Elizabeth | + | + |
| 11. | Ice berg | + | + |
| 12. | Virgo | + | + |
| 13. | First prize | + | + |
| 14. | Sonia | + | + |
| 15. | Raktgandha | + | ++ |
| 16. | Ganga | + | + |
| 17. | Arabian night | + | + |
| 18. | Landora | + | + |
| 19. | Pink panther | + | + |
| 20. | Madhura | − | − |
| 21. | Pusa virangana | − | − |
| 22. | Keshar fire | + | + |
| 23. | Pusa manhar | + | + |
| 24. | Ahylia | − | − |
| 25. | Ephil tower | + | + |
| 26. | Lusiana | − | − |
| 27. | Pusa baramasi | − | − |
| 28. | Sandigum santinari | − | − |
| 29. | Arjun | + | + |

+ strong reaction,
++ very strong reaction,
− negative reaction

### EXAMPLE 2

Raising of Antisera:

Purified recombinant coat protein was used an antigen for immunization of rabbit. Healthy white New Zealander male albino rabbits approximately six months old were used to raise the hyperimmune sera against PNRSV. Antigen (about 100 μg per injection) was mixed with Freund's adjuvant in the ratio of 1:1 and injected by two routes intramuscularly and subcutaneously into the thigh muscles of rabbits.

First two injections were given along with Freund's complete adjuvant at the interval of one week. Similarly second and third injections were given along with Freund's incomplete adjuvant (1:1) at the interval of one week. After two-week immunization schedule, the animals were bled from the marginal ear vein. The blood was collected in a glass tube and allowed to clot at room temperature for an hour. Subsequently, glass tube containing clotted blood was kept at 4° C. overnight. The serum was collected using pasture pipette and centrifuged at 5000 rpm for ten min at 2-6° C. The supernatant was collected and stored at 4° C. after adding sodium azide to a concentration of 0.2% (w/v). To collect more serum, booster injections were given 5, 12, 16 and 22 weeks after the initial injection.

For reference and serological testing, antiserum for PNRSV was procured from BioRad, USA.

Purification of Antibody (Separation of IgG from Whole Serum): A) By Ammonium Sulphate Precipitation:

1. Distilled water (9 ml) was added to 1 ml of crude antiserum.
2. Slowly drop wise 10 ml of neutralized saturated ammonium sulphate (Sigma) was added and continuously kept under stirring.
3. After stirring, it was kept at room temperature for about 1 hour. The resulting solution should appear viscous and cloudy because of precipitation of antibodies i.e. IgG.
4. Solution was centrifuged at 9000 g for 15 min and precipitate was washed with 2 ml of half-strength PBS. Washing step was repeated three times to remove the traces of ammonium sulphate.
5. Finally precipitate was dissolved in 1 ml of half strength PBS.
6. O.D. was measured at a wavelength of 280 nm.
7. The antibodies were diluted in a way that final concentration became 1 mg/ml (O.D. reading 1.4=1 mg/ml).
8. 1 ml aliquots along with 0.02% w/v sodium azide were stored at −20° C. for further use. PBS (100 ml): Na2HPO4.12H2O=5.8 gm; NaH2PO4.2H2O=1.0 gm; NaCl=8.76 gm.
   B) By Affinity Chromatography:
1. Protein A-sepharose (Sigma) was swelled and packed in a column.
2. Column was washed with equilibration buffer.
3. Serum was diluted and passed through the column with a regulated flow.
4. Unbound proteins were washed with PBS until no more protein leaves the column (it was monitored by spectrophotometer).
5. Bound protein (IgG) was eluted with the elution buffer.
6. pH was neutralized with Tris HCl.
7. Column was regenerated by washing alternatively with equilibration buffer and storage buffer. Then the column was stored in storage buffer at 4° C.
8. Elute was dialyzed thrice against PBS and stored at −20° C. until used further. PBS (100 ml): Na2HPO4.12H2O=5.8 gm; NaH2PO4.2H2O=1.0 gm; NaCl=8.76 gm.
   Equilibration buffer (5×): Tris—0.05 M; NaCl—0.15 M, pH 8.6.
   Storage buffer: Na2HPO4—0.05 M; Thomersol—0.05%, pH 6.0
   Elution buffer: CH3COONa—0.05 M; NaCl—0.15 M, pH 4.5
      Preparation of Antibody Enzyme Conjugate (Using Alkaline Phosphatase):
1. 1 mg of alkaline phosphatase (Sigma) was dissolved in 2 ml of purified antibodies.
2. Fresh gluteraldehyde (25% stock, Merck) was added to the solution in such a way to make the final concentration 0.05% and mixed well.
3. It was incubated at room temperature for 4 hrs. A faint brown colour was developed.
4. After 4 hrs, it was centrifuged at 9000 g for 20 min.
5. The precipitate was washed twice with half strength PBS and finally dissolved in 2 ml of half strength PBS.
6. Bovine serum albumin (BSA) to 5 mg/ml and sodium azide to 0.02% w/v were dissolved in it to enhance its self life. It was stored at 4° C. till further use.
   Evaluation of Alkaline Phosphatase Conjugate:

Activity of conjugate was checked by DAS-ELISA as described in the examples given in complete specifications of patent using known positive and negative samples and titrated too.

### EXAMPLE 3

Detection of PNRSV from Rubus spp:

To detect the PNRSV, two different species (Rubus ellipticus and Rubus navalis) of Rubus spp. were checked using DAS-ELISA as described above. Extraction of sample was similar to the steps used for Rose sample as given in example 1. At the same time they were also checked by a reference kit DSMZ (Deutsche Sammlung von Mikroorganismen und Zellkulturen GmbH, Germany) and the results are summarized in Table 2.

TABLE 2
ELISA results of different species of Rubus spp. using reference
kit and test kit.

| | | ELISA using | |
|---|---|---|---|
| S.No. | Cultivars of Rubus | Reference kit | ELISA using test kit |
| 1. | Rubus ellipticus | + | ++ |
| 2. | Rubus navalia | + | ++ |

+ strong reaction,
++ very strong reaction,
− negative reaction

### EXAMPLE 4

Detection of PNRSV from Hop: To detect the PNRSV from hop by using the kit, samples were checked using DAS-ELISA as described above. Extraction of sample was similar to the steps used for Rose samples as given in example 1. At the same time, they were also checked by a reference kit DSMZ (Deutsche Sammlung von Mikroorganismen und Zellkulturen GmbH, Germany) and results are depicted in Table 3.

TABLE 3
ELISA results of hop using reference kit test kit

| | | ELISA using | |
|---|---|---|---|
| S.No. | Cultivar of Hop | Reference kit | ELISA using test kit |
| 1. | Humulus (Hop) | + | + |

### EXAMPLE 5

Detection of PNRSV from Stone Fruit and Other Crops:

To detect the PNRSV from stone fruit and other floriculture/ornamental crops, samples were checked using DAS-ELISA as described above. Extraction of samples was similar to the steps used for Rose samples as given in examples 1. At the same time they were also checked by a reference kit DSMZ (Deutsche Sammlung von Mikroorganismen und Zellkulturen GmbH, Germnany) and results are depicted in Table 4.

TABLE 4
ELISA results of stone fruits and other floriculture/ornamental
crops using reference kit ant test kit.

| | | ELISA using | |
|---|---|---|---|
| S.No. | Crops | Reference kit | ELISA using test kit |
| 1. | Apple (golden) | + | + |
| 2. | Apple | + | + |
| 3. | Peach cerasus | + | + |
| 4. | Peach persica | + | + |
| 5. | Peach (plum) | + | + |
| 6. | Peach (almond) | + | + |
| 7. | Begonia | + | + |
| 8. | Geranium | + | + |

### EXAMPLE 6

Preparation of Kit for Detection of Coat Protein of Prunus Necrotic Ringspot Virus

Said kit is prepared by assembling following ingredients along with an instruction manual. The methodology to prepare the following ingredients has already been mention in previous examples. The kit comprising the following:
a) polyclonal antibodies against Prunus necrotic ringspot virus coat protein in plants;
g) conjugate labeled with alkaline phosphatase;
h) coating buffer;
i) extraction buffer;
j) ECI buffer;
k) PNP buffer.
   Advantages:

The main advantages/applications of the present invention are
1. Rose is one of the choices of cut flower growers of India and abroad. Since it severely gets affected by PNRSV which reduces its vigor in the subsequent years, to develop the disease free propagation material and selection of healthy plants by using indigenous diagnostic kit are absolutely essential as it helps in the plant virus management.
2. The kit can detect all the PNRSV strains including Indian.
3. This being an indigenous diagnostic kit is cost effective too.
4. Since it is based on the polyclonal antibodies, it also detects other strains of PNRSV infecting other crops.
5. The kit can detect PNRSV from floriculture crops like rose (Garden roses, cut flower roses and essential oil bearing roses), wild plants (Rubus spp.), stone fruit crops (apple, peach, plum, apricot, almond and cherry), and other crops like hop, cucumber, begonia and geranium.
6. The components of the kit can be used to detect PNRSV in TCP's of commercial crops.
7. The components of the kit can be used in quality plant production programme.
8. The components of the kit can be used in plant quarantine purposes.
9. The cloned/expressed coat protein of PNRSV is a regular supply of coat protein of PNRSV for the production of antibodies against PNRSV.
10. The components of the kit give better signal (as in case of rose and Rubus spp.) as compared to the reference kit.
11. The components of the kit can be used for disease epidemiology and disease forecasting.
12. The development of the kit will ensure proactive measures for more effective check and the spread of virus.

We claim:

1. Primers useful for detection of Prunus necrotic ringspot virus (PNRSV) in plants, comprising the following sequence:
Sequence ID 1: upstream primer AACTGCAGATGGTTTGCCGAATTTGCAA
Sequence ID 2: downstream primer GCTCTAGACTAGATCTCAAGCAGGTC

2. A method for detection of Prunus necrotic ringspot virus (PNRSV) in plants, wherein the said method comprising the steps of:
a) providing a purified coat protein of PNRSV by using designed primers of Sequence ID 1: upstream primer AACTGCAGATGGTTTGCCGAATTTGCAA Sequence ID 2: downstream primer GCTCTAGACTAGATCTCAAGCAGGTC;
b) preparing polyclonal antibodies against PNRSV coat protein obtained from step (a); c) performing direct antibody sandwich enzyme linked immunosorbent assay (DAS ELISA) for detection of PNRSV.

3. A method as claimed in claim 2(a), wherein the complete coat protein of PNRSV is amplified from rose using designed primers having a Sequence ID 1: upstream primer AACTGCAGATGGTTTGCCGAATTTGCAA Sequence ID 2: downstream primer GCTCTAGACTAGATCTCAAGCAGGTC

4. A method as claimed in claim 2(a), wherein the complete coat protein of PNRSV with sequence ID 3:
MVCRICNHTHAGGCRSCKKCHPNDALVPLRAQQRVANNPNRNRNPNRVSSGM GPAVRPQPVVKTTWTVRGPNVPPRIPKGYVAHNHREVMTTEAVKYLSIDFTTTL PQLMGQNLTLLTVIVRMNSMSSNGWIGMVEDYKVDQPDGPNALSRKGFLKDQP RGWQFEPPSDLDFDTFARTHRVVIEFKTEVPAGAKVLVRDLYVVVSDLPRVQIPT DVLLVDEDLLEI is cloned in pGEX-2TK followed by transformation using E-coli strain BL 21.

5. A method as claimed in claim 2(a), wherein the optimal expression of PNRSV coat protein is checked with 0.5-0.9 mM IPTG concentration at about temperature 30 degree C. for 3-3.5 h.

6. A method as claimed in claim 2(a), wherein the purification of PNRSV coat protein is carried out by the known method.

7. A method as claimed in claim 2(b), wherein the immunization in rabbits are carried out three times with purified coat protein of PNRSV obtained from step 1(a) and Freund's complete adjuvant in the ratio of 1:1 at weekly intervals.

8. A method as claimed in claim 2(b), wherein the route for immunization may be intramuscularly, subcutaneously or intravenously.

9. A method as claimed in claim 2(b), wherein the rabbits are bled after 14 to 15 days to obtain polyclonal antibodies against PNRSV coat protein.

10. A method as claimed in claim 2(b), wherein the polyclonal antibodies against PNRSV coat protein are purified from the serum by known methods.

11. A method as claimed in claim 2(c), wherein the microtiter plates are coated with polyclonal antibodies diluting in a coating buffer in a ratio ranges from 1:500-1:1000 followed by 4-5 times washing with PBS-T.

12. A method as claimed in claim 2(c), wherein the test samples are prepared in microtiter plates by macerating infected leaf tissue from plant with extraction buffer followed by dilution from 1×- 1/160× of the original antigen.

13. A method as claimed in claim 2(c), wherein the microtiter plate is incubated overnight at about 37° C. followed by washing to allow coating of antigen in the wells.

14. A method as claimed in claim 2(c), wherein antibody conjugate obtained from step 1(b) in ECI buffer is added in the ratio ranges between 1:500 to 1:1000 for a period of about 4 hrs at about 37° C. followed by washing with PBS-T.

15. A method as claimed in claim 2(c), wherein about 100 μl of about 1 mg/ml p-nitrophenyl phosphate solution in PNP buffer is added in the mix as claimed in claim 14.

16. A method as claimed in claim 2(c), wherein the reaction is terminated by adding about 50 μl of about 3M NaOH after 15-20 min to obtain yellow color product.

17. A method as claimed in claim 1(c), wherein the color product is antigen and antibody conjugate.

18. A method as claimed in claim 2(c), wherein absorbance of colored product is measured at 405 nm for detection of Prunus necrotic ringspot virus.

19. A diagnostic kit useful for detection of coat protein of Prunus necrotic ringspot virus comprising:
a) polyclonal antibodies against Prunus necrotic ringspot virus coat protein in plants as claimed in claim 2;
b) conjugate labeled with alkaline phosphatase;
c) coating buffer;
d) extraction buffer;
e) ECI buffer;
f) PNP buffer.


SEQUENCE LISTING (3 sequences)

SEQ ID NO: 1 - DNA, 28 bp, Prunus necrotic ringspot virus
aactgcagatggtttgccgaatttgcaa

SEQ ID NO: 2 - DNA, 26 bp, Prunus necrotic ringspot virus
gctctagactagatctcaagcaggtc

SEQ ID NO: 3 - protein, 226 aa, Prunus necrotic ringspot virus
MVCRICNHTHAGGCRSCKKCHPNDALVPLRAQQRVANNPNRNRNPNRVSSGMGPAVRPQPVVKTTWTVRGPNVPPRIPKGYVAHNHREVMTTEAVKYLSIDFTTTLPQLMGQNLTLLTVIVRMNSMSSNGWIGMVEDYKVDQPDGPNALSRKGFLKDQPRGWQFEPPSDLDFDTFARTHRVVIEFKTEVPAGAKVLVRDLYVVVSDLPRVQIPTDVLLVDEDLLEI